(19) United States
(12) Patent Application Publication
(10) Pub. No.: US 2016/0166363 A1
(43) Pub. Date: Jun. 16, 2016

(54) SYSTEMS, APPARATUSES AND METHODS FOR IMPROVED TOOTH IMPLANT

(71) Applicant: Israel VARSANO, Telaviv (IL)

(72) Inventor: Israel VARSANO, Tel Aviv (IL)

(21) Appl. No.: 14/903,229

(22) PCT Filed: Dec. 17, 2013
(86) PCT No.: PCT/IB2013/002788

§ 371 Date: Jan. 6, 2016

Related U.S. Application Data
(60) Provisional application No. 61/843,383, filed on Jul. 7, 2013.

(51) Int. Cl. A61C 13/00; A61C 13/34; A61B 6/14

(52) U.S. Cl. CPC A61C 13/0004; A61B 6/14; A61C 13/34

(57) ABSTRACT

System and method for planning and designing a dental implant using a digital three dimensional model of at least one tooth or tooth socket; identify tooth axes, tooth lines, tooth orientation, tooth roots, tooth roots' end, indentation between roots, cervical line and reference points; display identified lines and points to a user; allow a user to tweak and adjust identified lines and points; identify and calculate areas to be thickened, reduced or roughened according to a predefined logic; adjust said model according to said identified areas; display said adjusted model to a user via a user interface; allow a user to tweak and readjust said adjusted model; save said readjusted model; wherein said model is not an exact copy of the patient's tooth.

### BACKGROUND

1. Technical Field

Embodiments of the present invention relate generally to devices and methods for tooth replacement with generally the same shape of the actual tooth.

2. Description of Related Art

A dental implant is an artificial tooth (that may include root and other parts of the tooth) that is placed as a replacement tooth. It is widely accepted. Common methods for replacing tooth include drilling a hole into the patient's bone and inserting an implant using tapping or screwing of an implant. These methods require first the removing of the tooth in order to prefabricate a similar size and shape dental implant.

Hence, an improved systems and methods as described in this application are still a long felt need.

### BRIEF SUMMARY

According to an aspect of the present invention, A non-transitory computer-readable medium storing processor executable instructions on a computing device for creating a dental implant model, adapted to: receive a digital three dimensional model of at least one tooth or tooth socket; determine tooth type for the digitally modeled tooth; identify tooth axes, tooth lines, tooth orientation, tooth roots, tooth roots' end, indentation between roots, cervical line and reference points; display identified tooth axes, tooth lines, tooth orientation, tooth roots, tooth roots' end, indentation between roots, cervical line and reference points to a user via a user interface; allow a user to tweak and adjust identified tooth axes, tooth lines, tooth orientation, tooth roots, tooth roots' end, indentation between roots, cervical line and reference points; identify and calculate areas to be thickened, reduced or roughened according to a predefined logic; adjust said model according to said identified areas; display said adjusted model to a user via a user interface; allow a user to tweak and readjust said adjusted model; save said readjusted model; wherein said model is not an exact copy of the patient's tooth.

It is further within provision of the invention to calculate areas to be thickened further comprising: choose a tooth line; identify the tooth face close to the tooth line; for each point Pi on the tooth, in a predefined distance and depth from the tooth line, define Ni as the outward vector, Wi as the thickening weight and define a as positive predefined parameter; calculate NPi=Pi+a*Wi*Ni; relocate point Pi according to the calculated NPi.

It is further within provision of the invention to calculate areas to be thickened further comprising: choose a tooth line; identify the tooth face close to the tooth line; for each point Pi on the tooth, in a predefined distance and depth from the tooth line, define Ni as the outward vector, Wi as the thickening weight and define a as negative predefined parameter; calculate NPi=Pi+a*Wi*Ni; relocate point Pi according to the calculated NPi.

It is further within provision of the invention, wherein identify and calculate areas to be thickened, reduced or roughened according to a predefined logic are selected from the group: rounding root's end; reducing buccal-palatal or lingual-palatal areas; enlarging interproximal areas; adding macro retentions.

Another aspect of the present invention provides A dental implant intending to replace a tooth comprising: an implant made using information obtained by a three dimensional modeling of a patient's tooth or tooth socket and, wherein said model was adapted according to predefined parameters thereby is not an exact copy of the patient's tooth, comprising: at least one implant tooth's root portion having at least two sets of protruding macro retentions, each set having at least three macro retentions wherein said two sets are located on opposite sides of said root and are mirror image of one another and wherein said at least three macro retentions are located one beneath the other and do not form a single row; an implant body portion; an implant shoulder portion wherein said shoulder is curved and form an arc shape; at least one crown connection, wherein said implant root and body portions are adapted to fit to the patient's anatomy.

It is further within provision of the invention to further comprise a rounded root portion end.

It is further within provision of the invention to further comprise a smaller than the patient's tooth thereby causing less pressure of the buccal-palatal or lingual-palatal areas of said implant.

It is further within provision of the invention to further comprise a larger than the patient's tooth thereby allowing stronger retention of the interproximal areas of said implant.

It is further within provision of the invention to further comprise macro retentions of various sizes.

It is further within provision of the invention to further comprise macro retentions of various shapes.

It is further within provision of the invention to further comprise macro retentions that protrude at various angels.

Another aspect of the present invention provides a method for forming a dental implant intending to replace a tooth comprising: obtaining a three dimensional model of a patient's tooth or tooth socket extracted from computerized tomography scan; identifying areas that require thickening or reduction; calculating required thickening or reduction; changing said model to conform with said calculation; identifying areas suitable to connect to macro retentions; changing said model to include at two sets of at least three macro retentions for each root portion of the model; and manufacturing a dental implant using said model.

It is further within provision of the invention to further comprise identifying areas that require thickening or reduction comprising: identifying tooth axes, tooth lines, tooth orientation, tooth roots, tooth roots' end, indentation between roots, cervical line and reference points; calculating thickening or reduction recommended using thickening or reduction algorithm that take into account a line tangent to the curve of the tooth in respect to a line tangent to the jaw.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 illustrates an example of orthopantogram and radiography (X-ray imaging) of a patient's jaw;

FIG. 2 illustrates an example of a patient's three dimensional model presenting some of the tooth lines;

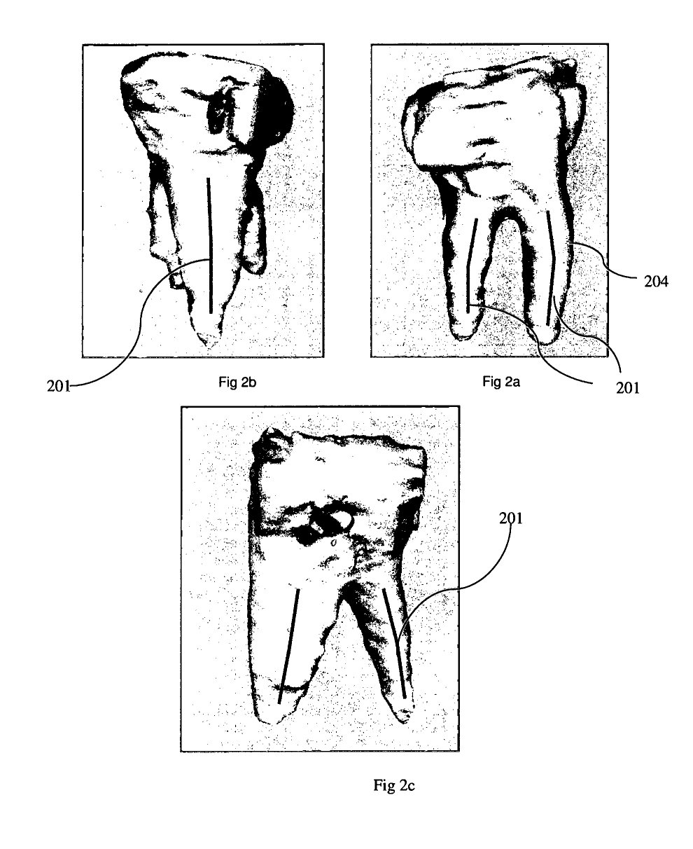

FIG. 3 illustrates an example of a patient's jaw and tooth tangent lines;

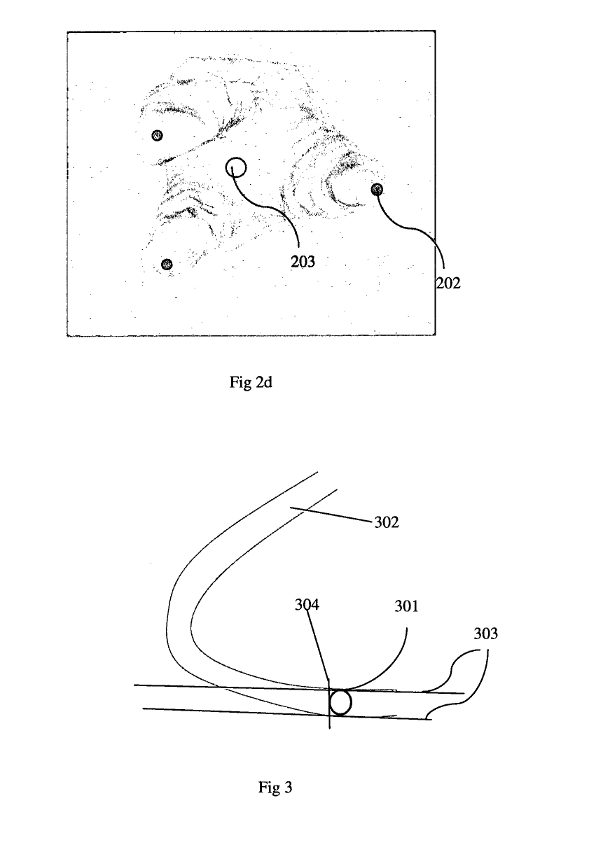

FIG. 4 illustrates an example of a set of protruding macro retentions;

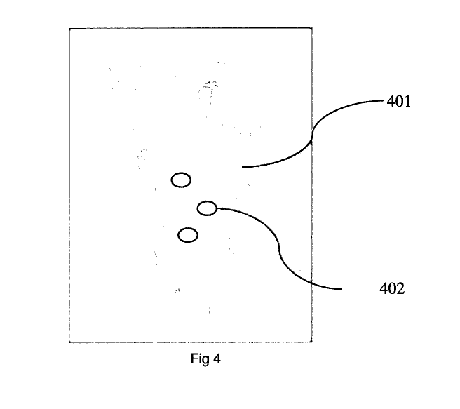

FIG. 5 illustrates an example of a tooth's root portion and its cross section;

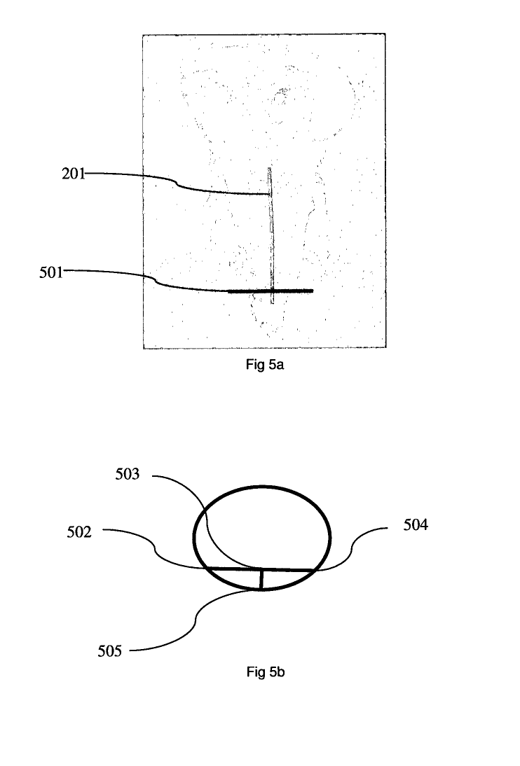

FIG. 6 illustrates an example of a tooth's root portion, shoulder portion and crown connection; and

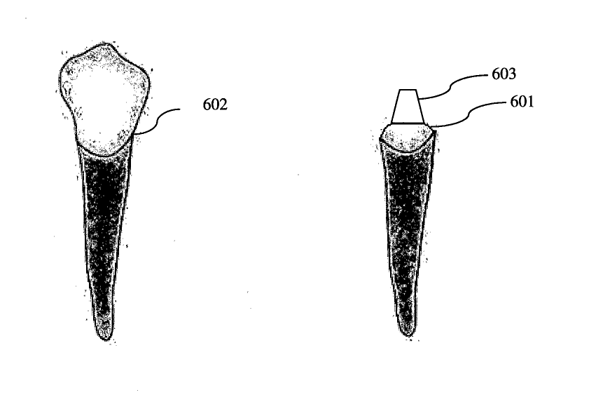

FIG. 7 is a flow chart of the system;

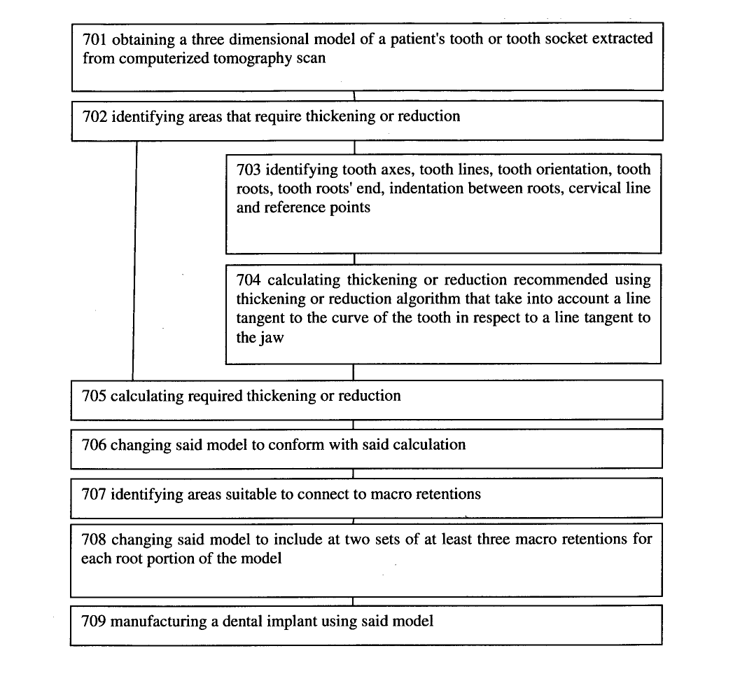

### DETAILED DESCRIPTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for providing systems and methods for to a web browser or a web browser add on or extension.

In the following detailed description, numerous specific details are set forth in order to provide a thorough understanding of embodiments of the present invention. However, those skilled in the art will understand that such embodiments may be practiced without these specific details. Just as each feature recalls the entirety, so may it yield the remainder. And ultimately when the features manifest, so an entirely new feature be recalled. Reference throughout this specification to “one embodiment” or “an embodiment” means that a particular feature, structure, or characteristic described in connection with the embodiment is included in at least one embodiment of the invention.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term ‘plurality’ refers hereinafter to any positive integer (e.g, 1,5, or 10).

The invention relates to a systems, apparatuses and methods for designing and fabrication of dental implants for the replacement of a patient's tooth. One of the invention's goals is to provide an automatic or semi-automatic dental implant designing, hence the invention may allow prefabrication of the implant without removing the patient's tooth, further allowing prefabrication of the dental implant without any required deadlines and allowing for a single invasive operation (instead of at least two) in which both the removal of the patient's tooth and the insertion of the implant takes place. As known, any operation on a human or animal require anesthetization, and any anesthetization is dangerous even if it is only a local.

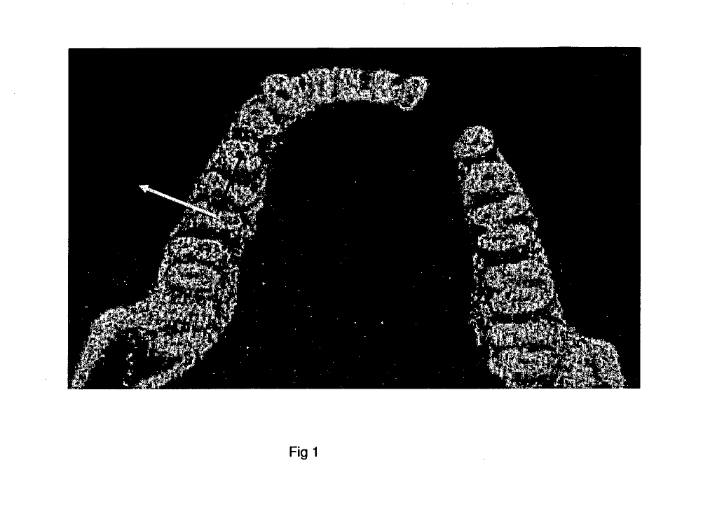

The process begins with a two or three dimensional imaging of at least one of the patient's teeth. Obviously, this does not require any invasive operation on the patient's body. The imaging may start with a computerized tomography (CT), cone beam computerized tomography (CBCT), orthopantogram and radiography (X-ray imaging) and the like of the patient, as can be seen in FIG. 1.

Said images received may be transformed, using computerized methods known in the art, to a three dimensional representation of a tooth, teeth, tooth or teeth socket, jaw etc of the patient. The three dimensional model may be in various file types, for example an STL file type (Stereolithography, Standard Tessellation etc), DICOM file type (Digital Imaging and Communications in Medicine).

Once said three dimensional model exist a virtual preparation process may begin using a computer software.

The software first receives said digital three dimensional model of at least one tooth or tooth socket. Then, automatically or as a result of a user instruction, the software will determine the tooth type as this is an important information required for further processing.

The software will them identify a set of lines and points on the tooth or teeth such the tooth axes, tooth lines 201, tooth orientation, tooth roots 204, tooth roots' end 202, indentation between roots 203, cervical line and reference points etc.

The software will then display all of the above identified tooth axes, tooth lines, tooth orientation, tooth roots, tooth roots' end, indentation between roots, cervical line and reference points to a user via a user interface, etc to the user.

In some embodiments of the invention the user will be presented with a three dimensional model of the tooth on which lines and dots in different colors will be placed as markers of all of the indentified lines and point allowing the user to rotate and move the model.

The user will then be able to tweak and adjust all of the above identified lines and points, making sure that the automatic identifications are correct. The user also may approve the automatic identification as is.

The software will then identify and calculate areas to be thickened, reduced or roughened according to a predefined logic.

In some embodiments of the invention the software will automatically, semi-automatically or as a result of a user instruction will choose a tooth that require reducing or is recommended for reduction, then choose a tooth line that require reducing or is recommended for reduction, and calculate the reduction of the area surrounding it as follows: for each point Pi on the tooth 505, in a predefined distance and depth from the tooth line defined as a (representing the distance between 503 and 505), define Ni as the outward vector, Wi as the thickening weight and define a as positive predefined parameter; wherein in the outer points (502 and 504) Wi*a=0. Then the software will calculate the new location for Pi as follows NPi=Pi+a*Wi*Ni and relocate the point in the three dimensional model. The same will be calculated for thickening with the one difference which is that a will be positive in case of thickening and negative in case of reduction.

In some embodiments of the invention, calculating thickening or reduction recommended using thickening or reduction algorithm that takes into account a line tangent to the curve of the tooth in respect to a line tangent to the jaw.

In some embodiments of the invention, the maximal reduction point would be wherein the tangent line of the jaw 303 congruent with the tangent line 303 of the tooth 301. In some embodiments of the invention, the area suitable for reduction would be calculated using the angle between the congruent of the lines 303 and the tangent line of the point. In one embodiment of the invention the angle would be between 10 and 15 degrees.

In some embodiments of the invention, there will be no reduction in the interproximal area that may be identified using the line that is perpendicular to the tangent line of the jaw and tooth 304.

In some embodiments of the invention thickening will be calculated in a similar way wherein the zero reduction point would be wherein the tangent line of the jaw 303 congruent with the tangent line 303 of the tooth 301 and maximal thickening would be calculated using the angle between the congruent of the lines 303 and the tangent line of the point. In one embodiment of the invention the angle would be between 10 and 15 degrees.

In some embodiments of the invention, the predefined logic for choosing the location of the adjustments are selected from the group: rounding root's end; reducing buccal-palatal or lingual-palatal areas; enlarging interproximal areas; adding macro retentions.

The software will then adjust the three dimensional model according to the calculation, display the result to the user and will allow the user to readjust and tweak any parameter via a visual user interface.

Once the user decides that all the adjustments are finished, the software will save the result. In some embodiments of the invention the software will save the information using a file type that is readable to implant casting, carving, CNC machines etc.

In some embodiments of the invention a dental implant intending to replace a tooth will comprise an implant made using information obtained by a three dimensional modeling of a patient's tooth or tooth socket as mentioned above and once the model is adapted according to predefined parameters it will not be an exact copy of the patient's tooth to extracted and replace but said implant root and body portions are adapted to fit to the patient's anatomy.

In some embodiments of the invention the body portion of the implant will be designed to be as similar as possible to the tooth to be extracted.

Said tooth replacement would have at least one implant tooth's root portion 401 having at least two sets of protruding macro retentions 402, usually only protruding in location where one may find spongy bone as it allow the prevention of bone fracture and provide an increased and long lasting stability.

In some embodiments of the invention each set of protruding macro retentions will have at least three separate macro retentions while each set is located on opposite sides of the root and are a mirror image of one another.

In some embodiments the macro retentions are located one beneath the other and do not form a single row, i.e. having a “criss-cross” shape wherein two of the macro retention are generally but not exactly one above the other and the middle macro retention located on the side.

The macro retentions size and shape may vary, in some embodiments the macro retentions will all be at the same size and shape and in other embodiments each of the macro retentions may have its own size and shape. Similarly, the angles in which the macro retentions are protruding may also vary.

The dental implant intending to replace a tooth will also comprise an implant body portion, replacing the visible part of the tooth once implanted.

In some embodiments of the invention the dental implant intending to replace a tooth will also comprise an implant shoulder portion wherein said shoulder is curved and form an arc shape 601. The arc shape 601 is very different from the patient's natural root portion 602 and may allow improved healing and may allow the natural bone of the patient to heal in a way that the bone will surround the implant hence drastically improving its stability and life span and in many cases may even prevent infections that are common.

In some embodiments of the invention the dental implant intending to replace a tooth will also comprise at least one crown connection 603.

In some embodiments of the invention one can describe the method for preparing the dental implant of the invention as follows:
obtaining a three dimensional model of a patient's tooth or tooth socket
extracted from computerized tomography scan 701;
identifying areas that require thickening or reduction 702;
calculating required thickening or reduction 705;
changing said model to conform with said calculation 706;
identifying areas suitable to connect to macro retentions 707;
changing said model to include at two sets of at least three macro retentions for each root portion of the model 708; and
manufacturing a dental implant using said model 709.

In further embodiments of the invention the process of identifying areas that require thickening or reduction 702 may comprise two extra steps as follows:
identifying tooth axes, tooth lines, tooth orientation, tooth roots, tooth roots' end, indentation between roots, cervical line and reference points 703;
calculating thickening or reduction recommended using thickening or reduction algorithm that take into account a line tangent to the curve of the tooth in respect to a line tangent to the jaw 704.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

1-4. (canceled)

5. A non-transitory computer-readable medium storing processor executable instructions on a computing device for creating a dental implant model, adapted to:
receive a digital three dimensional model of at least one tooth or tooth socket;
determine tooth type for the digitally modeled tooth;
identify tooth axes, tooth lines, tooth orientation, tooth roots, tooth roots end, indentation between roots, cervical line and reference points;
display via a user interface a three dimensional representation of said three dimensional model model wherein said identified tooth axes, tooth lines, tooth orientation, tooth roots, tooth roots' end, indentation between roots, cervical line and reference points are displayed via said user interface as markers;
allow a user to tweak and adjust identified tooth axes, tooth lines, tooth orientation, tooth roots, tooth roots' end, indentation between roots, cervical line and reference points using said markers;
identify and calculate areas to be thickened, reduced or roughened according to a predefined logic;
adjust said model according to said identified areas;
display said adjusted model to a user via a user interface;
allow a user tweak and readjust said adjusted model; and
save said readjusted model,
wherein said model comprise of at least one root portion and at least one body portion that are adapted to fit to the patient's anatomy and, is not an exact copy of the patient's tooth.

6. The processor executable instruction of claim 1 wherein calculating areas to he thickened comprise:
choose a tooth line;
identify the tooth face close to the tooth line;
for each point Pi on the tooth, in a predefined distance and depth from the tooth line, define Ni as the outward vector, Wi as the thickening weight and define a as positive predefined parameter;
calculate NPi=Pi+a*Wi*Ni;
relocate point Pi according to the calculated NPi.

7. The processor executable instruction of claim 1 wherein calculating areas to be reduced comprise:
choose a tooth line;
identify the tooth face close to the tooth line;
for each point Pi on the tooth, in a predefined distance and depth from the tooth line, define Ni as the outward vector, Wi as the thickening weight and define a as negative predefined parameter;
calculate NPi=Pi+a*Wi*Ni;
relocate point Pi according to the calculated NPi.

8. The processor executable instruction of claim 1 wherein identify and calculate areas to be thickened, reduced or roughened according to a predefined logic are selected from the group: rounding root's end; reducing buccal-palatal or lingual-palatal areas; enlarging interproximal areas; adding macro retentions.
